(19) United States
(12) Patent Application Publication
(10) Pub. No.: US 2022/0409405 A1
(43) Pub. Date: Dec. 29, 2022

(54) PROSTHESIS COVER FOR A PROSTHESIS, PARTICULARLY FOR A PROSTHETIC LEG

(71) Applicant: Romedis GMBH, Neubeuern (DE)

(72) Inventor: Andreas RADSPIELER, Neubeuern (DE)

(21) Appl. No.: 17/763,580

(22) PCT Filed: Sep. 16, 2020
(86) PCT No.: PCT/EP2020/075830

§ 371 Date: Mar. 24, 2022

(30) Foreign Application Priority Data
Sep. 25, 2019 (DE) 10 2019 125 870.7
Jul. 27, 2020 (DE) 10 2020 119 762.4

(51) Int. Cl. A61F 2/78

(52) U.S. Cl. CPC A61F 2/7812; A61F 2002/7837; A61F 2/60

(57) ABSTRACT

The present invention relates to a prosthesis covering with a closed cross-section which comprises a first section, a second section and a third section. The second section is arranged in longitudinal direction between the first section and the third section and comprises other material, other diameter and/or other circumference than one or both of the other two sections.

### FIELD OF THE INVENTION

The present invention relates to a prosthesis covering having a closed cross-section, i.e. to slip or to ascend with the extremity, for example the leg, thereinto.

### BACKGROUND OF THE INVENTION

Known from practice are prosthesis coverings which are placed on the skin and in particular over a patient's joint or prosthesis in order to bridge it.

### SUMMARY OF THE INVENTION

An object of the present invention is to suggest a further prosthesis covering and a prosthesis.

The object according to the present invention is achieved by a prosthesis covering and by a prosthesis with features as described herein.

Hereby, the prosthesis covering comprises along its longitudinal extension at least or exactly one first, second and third section or consists thereof. Thereby, the second section is arranged in longitudinal direction between the first and the third section.

The second section, e.g. in a second subsection thereof, optionally consists of or optionally comprises a material with a higher elasticity Modul (in short: E-Modul, alternatively: Young's modulus, alternatively defined as quotient of tension and stretching (o/c)) than the first and/or the third section or the materials of the first and/or of the third section.

In addition to or alternatively to the different elastic moduli mentioned above, the second section comprises a larger circumference and/or diameter than the first section and also than the third section.

During its intended use, the prosthesis covering serves to cover an extremity prosthesis, in particular a leg prosthesis, at least in sections. As intended, the prosthesis covering may rest, on the one hand, directly or separated by at least one material layer on the limb stump and, on the other hand, directly or separated by at least one material layer on a section of the prosthesis, for example a cosmetic supplement. Thereby, the prosthesis covering may bridge, cover or surround an artificial joint of the prosthesis.

The prosthesis according to the present invention, which may be designed in particular as a leg prosthesis or knee prosthesis, is at least partially covered or overlaid by a prosthesis covering according to the present invention.

The set according to the present invention comprises a prosthesis and a prosthesis covering according to the present invention.

In all of the following statements, the use of the expression “may be” or “may have”, and so on, is to be understood synonymously with “preferably is” or “preferably has”, and so on, respectively, and is intended to illustrate embodiments according to the present invention.

Embodiments according to the present invention may comprise one or several of the above-mentioned or following features. Thereby, the features mentioned herein may in any combination be subject-matter of embodiments according to the present invention unless the person skilled in the art recognizes a specific combination as technically impossible.

Embodiments according to the present invention are further described herein.

Whenever numerical words are mentioned herein, the person skilled in the art shall recognize or understand them as indications of a numerical lower limit. Unless it leads the person skilled in the art to an evident contradiction, the person skilled in the art shall comprehend the specification for example of “one” (or “a”) as encompassing “at least one”. This understanding is also equally encompassed by the present invention as the interpretation that a numerical word, for example, “one” (or “a”) may alternatively mean “exactly one”, wherever this is evidently technically possible for the person skilled in the art. Both understandings are encompassed by the present invention and apply herein to all used numerical words.

Whenever an embodiment is mentioned herein, it is then an exemplary embodiment according to the present invention.

Herein, the information “above” and “below” are to be understood, in case of doubt by the person skilled in the art, as absolute or relative spatial information which relates to the orientation of the component in question during its intended use.

When it is disclosed herein that the subject-matter according to the present invention comprises one or several features in a certain embodiment, it is also respectively disclosed herein that the subject-matter according to the present invention does, in other embodiments, likewise according to the present invention, explicitly not comprise this or these features, for example, in the sense of a disclaimer. Therefore, for every embodiment mentioned herein it applies that the converse embodiment, e.g. formulated as negation, is also disclosed.

In some embodiments, the second section comprises at least a first subsection which is tubular and connected to the first and/or to the third section, being likewise tubular, preferably in an air-tight manner.

In certain embodiments, the second section comprises at least one second subsection which extends in a band-like manner between the first and third section, particularly in a longitudinal direction of the prosthesis covering.

In some embodiments, the second section comprises the subsection which is referred to herein as the first section and/or the subsection which is referred to herein as the second section, or only one of these two, or consists of one or of both of these subsections.

In certain embodiments, the first subsection and the second subsection were made separately from each other and/or from different materials (or comprise such materials) before becoming part of the second section or of the prosthesis covering.

In some embodiments, the second section is folded or wrapped or comprises a further or ulterior reserve of material in at least one state thereof in at least its first subsection.

A prosthesis covering, as used herein, may also be a sealing covering, protective cover, vacuum(knee)bandage, cuff, prosthesis cuff, prosthesis liner. Interchanging these terms will lead in any case to combinations of features which are also encompassed by the present invention.

In several embodiments, the prosthesis covering is not a liner. It is optionally not intended to be worn under a prosthesis shaft. It is optionally intended to be worn over the prosthesis.

In some embodiments, the second section comprises at least one first subsection which is pleated/doubled over in at least one state of the prosthesis covering or lies in folds, waves and/or elevations or warps, or provides a material reserve in this or in other ways, preferably in a use state (i.e., dressed, i.e., used on the patient's or wearer's body) and/or in at least one non-use state (i.e., e.g., in its shipping package, lying unpacked on a table, etc.).

In other embodiments, the prosthesis covering and in particular the second section does not comprise a pleated/folded/doubled over subsection and/or bellows.

A doubled over subsection may be understood as a subsection in which material that forms the subsection or is involved in its formation, i.e. its wall or layer or layer thickness, is arranged at least in sections at least in two layers one above the other or one next to the other. Thereby, two layers of the material, that form the wall of the subsection, lie on top of each other or next to each other; this can e.g. be compared to a folding. Unfolding of the subsection may lead to a greater length (e.g. in a direction transverse to the direction of the fold) of the subsection. Thereby, the doubling over/folding over would be more or less dispensed with and would completely disappear when the subsection is completely unfolded. When unfolded completely, the subsection may only be single layer, depending on its design.

An example for a doubling over is known from the accordion, which comprises a bellows as one of the substantial components. In this, the width of the area in which the layers of the material lie in folds on top of each other usually extends transverse to the longitudinal direction of the bellows. Optionally, the width in which the layers of the material lie in folds on top of each other extends entirely, predominantly or substantially in the longitudinal direction and/or in the transverse direction or in the circumferential direction of the subsection or of the prosthesis covering.

In several embodiments, a layer of the wall is to be understood as the whole thickness of the, for example textile, wall. When the wall is folded, it then lies in layers on top of each other or next to each other. Layers, as used herein, may in several embodiments are not to be confused with the layers which make up the wall. A wall can comprise several layers, for example, an inner layer consisting of a very skin-friendly material or comprising such, and a very waterproof or airtight material that lies on top of the inner layer forming an outer layer. Thereby, when the wall is smoothed out, the outer and the inner layer form a common single layer. Only when the wall is folded, so that—from the inside to the outside—an inner layer, an outer layer, a further outer layer, an inner layer and finally an outer layer adjoin each other, would one refer herein to a wall as being in two layers or doubled over.

Above-mentioned statements referring to the inner and the outer layer may, in some embodiments, also be transferred to the inner or the outer side, respectively.

In several embodiments, the wall of the first, the second and/or the third section or subsections thereof, for instance the herein-described first and/or the second herein-described subsection of the second section, is made of exactly one layer.

In some embodiments, the second section comprises at least one first subsection. The descriptions of the first subsection made herein may apply in any combination to said first subsection.

In several embodiments, the second section comprises at least one second subsection. The descriptions of the second subsection made herein may apply in any combination to said second subsection.

In some embodiments, the second section comprises at least one first subsection which is folded/raised in at least one state of the prosthesis covering or in any state of its use.

In some embodiments, the second section comprises at least one first subsection which comprises in at least one state, for instance during use, of the prosthesis covering, a material reserve.

The term “material reserve” of a section may mean herein that the section comprises material or potential length which allows it to lie doubled, in folds, waves and/or warps, in particular, for example, to lie unstretched, such as in a longitudinal direction of the section and/or in a transverse or a circumferential direction of the section. Thus, an accordion (a musical instrument having a bellows which, by alternately compressing and expanding the bellows, generates air currents which cause reeds to vibrate and thus produce tones), which bellows is not fully expanded, would be considered, in the sense of the term “material reserve” as used herein, to have a material reserve in that said accordion or its bellows could be pulled further apart and thus elongated or stretched. It is obvious that the bellows has such a material reserve in most of the states it may assume. Only when it is completely pulled apart, so that it cannot be pulled further apart without having a destructive effect, it does no longer have such a material reserve.

However, in some embodiments a stretching in such a way that the material reserve of the first subsection would be used up in the longitudinal direction of the prosthesis covering, and the first subsection would thus be completely stretched, does not occur during the wearing of the prosthesis covering if the second subsection is provided. This is prevented by the second subsection which is shorter in the longitudinal direction of the prosthesis covering.

The term “material reserve” may be replaced by “length reserve” and/or “circumferential reserve” in some embodiments.

When reference is made herein to a section or to subsection having or not having a material reserve or length reserve, this may refer to a state in which the prosthesis covering is maximally stretched, for instance by hand, with light or moderate force with which the prosthesis covering is pulled apart or stretched in its longitudinal direction, as could be the case when the prosthesis covering is worn or put on as intended.

When reference is made herein to a section or to a subsection having or not having a material reserve or length reserve, then this may in some embodiments refer to a material reserve in the width or unwound. Material reserve may in several embodiments be provided in both the length and width, as well as in a circumferential direction of the prosthesis covering.

In some embodiments, the term “material reserve” may thus alternatively or additionally be replaced by “reserve in the width or unwound”.

In certain embodiments, the material of the first subsection and/or the material of the second subsection is not stretchable in the longitudinal direction of the prosthesis covering.

In some embodiments, the material of the first subsection is stretchable in the longitudinal direction and/or circumferential direction of the prosthesis covering. This allows the first subsection to already contact the leg/prosthesis after only a few steps with the prosthesis. This occurs without radial pressure or significant radial pressure and thus in a very pleasant way for the patient, because the first subsection, thanks to the second subsection, does not have to transmit any forces in the longitudinal direction of the prosthesis section, for instance when pulling over the prosthesis covering.

In several embodiments, the second section comprises at least one first subsection which is lamella-like in at least one state of the prosthesis covering.

In some embodiments, the second section comprises at least one first subsection which is completely or at least in sections multi-layer in at least one state of the prosthesis covering.

In several embodiments, the second section has at least one first area which, in at least one state of the prosthesis covering, is completely or at least in section multi-layer, while a second area, which is larger than the first area, is not multi-layer.

In further embodiments, the second section comprises at least one structure extending in longitudinal direction or at least also in longitudinal direction of the prosthesis covering. The structure is—preferably as such or in itself—optionally not folded, not doubled over, not curled up, not lamella-like or not multi-layer.

In some embodiments, this structure is the component of the second section, referred to herein as the second subsection. It may lie between, be connected to, and/or be disposed with the first and third sections, respectively, and may already limit or terminate the stretching of the prosthesis covering or of its second section, when attempting to maximally stretch the prosthesis covering in the longitudinal direction thereof, in the event that the first subsection would still be further stretchable. The second subsection may thus preferably significantly limit or determine the extent to which the first and third sections could move away from one another when stretching or expanding the prosthesis covering, for example when the patient pulls over or puts on the prosthesis covering. Further stretching of the prosthesis covering or of the second section, which would theoretically still be possible because the first subsection of the second section still has material reserve in the longitudinal direction or would be further stretchable due to the applied tensile forces which act on the prosthesis, for example, when the patient pulls the prosthesis covering over the prosthesis, may thus be prevented by the second subsection in several embodiments.

In several embodiments, this structure or the second subsection is or comprises at least one ribbon or at least one ribbon-like structure having a width between 0.5 cm and 10 cm, preferably between 1 cm and 7 cm, particularly preferably between 1 cm and 5 cm, between 1.5 cm and 5 cm, or between 1 cm and 3 cm.

In some embodiments, said structure or the second subsection is or comprises at least one ribbon or at least one ribbon-like structure which has a length corresponding to the distance between edges of the first section facing each other on the one hand and the third section on the other hand (e.g., at maximum expansion of the prosthesis cover), or is between 80% and 120% thereof, e.g. in an area of the prosthesis covering in which also the ribbon-like structure or the second subsection is arranged.

In some embodiments, the E-Modul of the material of this structure or of the second subsection is higher than the E-Modul of the first section and/or the third section, and/or less than or higher than the E-Modul of the material of the first subsection of the second section, preferably at least in a common direction, such as respectively in the longitudinal direction of the prosthesis covering.

In several embodiments, all or some of the lamellas, pleats wrappings, multi-layer sections or doubled over sections extend in circumferential direction, preferably not in the longitudinal direction, of the prosthesis covering nor of the second section.

In some embodiments, the second section comprises at least a first subsection which comprises the material reserve in the longitudinal direction of the second section or of the prosthesis covering.

In some embodiments, the first, second and/or third section of the prosthesis covering comprises or consists of an airtight material.

In several embodiments, the first, second, and/or third section of the prosthesis covering are joined together in an airtight manner, such as by gluing or textile welding.

In certain embodiments, the first subsection of the second section of the prosthesis covering comprises or consists of an airtight material. This material may extend optionally along the entire circumference of the second section.

In several embodiments, the first subsection and/or the second subsection of the second section of the prosthesis covering are not elastic.

In several embodiments, the first subsection, but not also the second subsection of the second section of the prosthesis covering is elastic.

In some embodiments, the first and/or the third section of the prosthesis covering are elastic.

In several embodiments, the term “non-stretchable” or “non-elastic” means that the elasticity modulus (Young's modulus), abbreviated as E-Modul, of the relevant subsection is at least higher than 200 N/mm², preferably higher than 700 N/mm², preferably higher than 1000 N/mm², most preferably higher than 2000 N/mm².

In certain embodiments, the term “non-stretchable” or “non-elastic” means that a stretching of the relevant subsection may be not more than 20%, preferably not more than 10%, preferably not more than 5%, most preferably not more than 2% of its length, before the subsection breaks.

In several embodiments, the relevant subsection or fibres thereof have an E-Modul the same as Nylon.

In certain embodiments, “stretchable” or “elastic” means the opposite of “non-stretchable” or respectively “non-elastic”. A fabric or a body is in these embodiments, therefore, either stretchable, or non-stretchable or respectively elastic or non-elastic.

In several embodiments the second section of the prosthesis covering comprises in at least one subsection thereof (e.g. in the first subsection) a, e.g. textile, elevated structure on its inner side, i.e. the side of the prosthesis covering or of the second section facing the body extremity while wearing the prosthesis covering.

The first subsection may optionally be made of a plastic (PP, PE, or other), a fiber-reinforced plastic, a composite material, a laminated material, a coated material, a coated textile or a fabric or may comprise such material.

The first subsection is preferably airtight or is made of, or comprises, an airtight material.

The second subsection may be made of a plastic (PP, PE, or other), a fiber-reinforced plastic, a composite material, a laminated material or a fabric or may comprise such material.

The first subsection and the second subsection differ optionally with regard to their materials.

The first section and/or the third section may be made of silicone, elastomer, silicone elastomer material, particularly a continuous, cured silicone elastomer material or may comprise such material.

In certain embodiments, the prosthesis covering or the second section provides a material reserve in the longitudinal direction of the prosthesis covering. The material reserve may optionally relate to the first subsection of the second section, if provided. It may be provided that the second subsection of the second section—if provided—has no material reserve.

The material reserve may be 10%, 20%, 30%, 40% or more, for example, when comparing the completely folded with the completely unfolded state. Therefore, a prosthesis covering or its second section may be extended/lengthened by unfolding, after separating the second section from the first and/or from the third section, e.g. by one of the above-mentioned percentages, or values in-between. Thereby, the unfolding is due preferably not or essentially not to elastic material properties, but rather to the unfolding or smoothing out, for example lamellas, pleats, waves, doublings, etc.

In some embodiments, the prosthesis covering does not comprise a closed end and thus may be referred to as tubular. In others, it has a closed end and thus may be referred to as sack-shaped.

In several embodiments, the prosthesis covering comprises one or more elasticized fabrics, and in others it does not, which may particularly apply for the second section or the first subsection of the second section.

In some embodiments, not every element or not every section of the prosthesis covering comprises elasticized fabrics, which may particularly apply for the second subsection of the second section.

In several embodiments, not each element or not each section of the prosthesis covering comprises fabrics or materials that have higher elasticity, E-Modul, or elastic stiffness in the one direction than in the other direction being orthogonal to that one direction.

In several embodiments, the prosthesis covering comprises fabrics or materials that have higher elasticity, E-Modul, or elastic stiffness in one direction or in the one direction than in the other direction being orthogonal to that direction. This may particularly apply for the first subsection of the second section.

In several embodiments, the prosthesis covering comprises fabrics or materials that have higher E-Modul in one direction or in the one direction than in the other direction being orthogonal to that direction. This may particularly apply for the first subsection of the second section.

In some embodiments, the inner surface of the prosthesis covering is not covered and/or not completely covered by a silicone elastomer material, in particular not with a continuously, hardened silicone elastomer material. In several embodiments the first and/or the third section are made of silicone or comprise silicone. In some embodiments the second section comprises no silicone or is not made of silicone.

In several embodiments, the second section of the prosthesis covering does not comprise a gel.

In some embodiments, the prosthesis covering does not comprise a gel.

In several embodiments, the prosthesis covering is not a joint bandage to be pulled over, does not consist of an elastic tube with a front side and a flexor side opposite to it, wherein the flexor side is at least in sections made of two layers, wherein the elastic tube is assembled from a front part and a rear part, wherein the rear part forms the flexor side.

In some embodiments, the prosthesis covering does not comprise, at its flexor side, flat-knitted sections between which tubular circular knitted fabrics are knitted in, which is arranged in the flexion area.

In several embodiments, the prosthesis covering is not a joint bandage with a tubular main body made of an elastic textile material with an insert in the flexion area, whereby the elasticity of the insert is higher than the elasticity of the main body.

In some embodiments, the prosthesis covering does not comprise an insert with two sections displaying different elasticity, the elasticities of which are each higher than the elasticity of the main body.

In several embodiments, the prosthesis covering is not a structure with surfaces made of textile threads having at least a transverse wave structure designed on one side, which is elastically pre-tensioned or stabilized by an elastic fabric arrangement that is integrated or arranged under a covering structure and which is connected to the covering structure with predetermined spacing.

In some embodiments, the prosthesis covering does not comprise a textile laminate with an elastic textile basis and a plastic coating arranged on it, the plastic coating being thinner than the textile basis and having a perforation integrated in order to adjust an adapted elasticity of the textile laminate.

In several embodiments, the prosthesis covering does not comprise any added pieces. This includes in particular attached, e.g. glued or sewn-on, parts, patches, pads or cushions. In certain embodiments, however, the prosthesis covering comprises a sealing tape which glues and/or seals, preferably on the inside, abutting edges running in the longitudinal direction of the second section and in particular of the first subsection thereof.

In some embodiments, the prosthesis covering is not a compression bandage, in particular not for wearing it on or over the healthy knee, above all, when pressure should be applied to the knee by a middle section of the prosthesis covering.

In several embodiments, the prosthesis covering does not comprise fabric grain orientations nor knit orientation, in particular no sections with different fabric grain/knit orientations.

In some embodiments, the prosthesis covering does not comprise protrusions on its inner side and/or its outer side.

In several embodiments, the prosthesis covering does not comprise, on its inner side and/or on its outer side, any material that is provided to prevent a movement of the prosthesis covering on the skin or of a layer arranged over the skin.

In several embodiments the prosthesis covering comprises, in its longitudinal direction, three or exactly three sections (e.g. the first, second and third section) which are connected to each other by gluing, stitching or by other means in order to form a tubular structure. Such gluing, stitching, or the like optionally extend only in the circumferential direction, optionally not in the longitudinal direction of the prosthesis covering, but at least one of the three sections is in turn glued, sewn, or differently connected to itself to form an annular or tubular structure. At least one of these sections may have, for example on its outer side or inner side, a structure as described herein as the second subsection of the second section.

Where reference is made herein that the first, second and third sections follow one another, e.g. by being glued, sewn or differently connected together to create a tubular structure, this may be the case, for example, on the outer skin or outer layer of the prosthesis covering, and the three sections may be distinguishable, for example, when looking at the prosthesis covering from the outside. In this, it is harmless for the structure divided into the first, second third section that, for example, on an inner side of the prosthesis covering, a gel or a fabric which may increase the wearing comfort of particularly the first and third section extends up to, for example, beyond a stitching between the first and second or third and second section, which may further increase the wearing comfort by covering the stitching.

In other embodiments, an outer skin extends across the first, second and/or third section and is manufactured, for example by a continuous material, such that the three sections are not distinguishable, e.g. when viewed from the outside of the prosthesis covering, or are not distinguishable by circumferential stitching, at least not when the three sections are covered by the outer skin, since circumferential stitching for connecting respective adjacent sections to each other is not provided, insofar it concerns the outer layer.

In several embodiments, the first, second and third section are each circumferential. Therefore, they each form a closed structure forming a through-opening into which the prosthesis and/or the stump of the patient is inserted, or through which the stump is guided.

In some embodiments, the first, second and third section are each air-impermeable circumferentially or in the radial direction.

In several embodiments, the prosthesis covering, the first, the second and/or the third section and/or the first subsection are each single-layer or have respectively single-layer sections.

In some embodiments, the first and/or third section is multi-layer, preferably having a first material, such as an elastic textile fabric, on its outer surface and a layer of other material underneath (on the inner surface), such as a gel or other material capable of providing the desired wearing comfort.

In several embodiments, the first and/or the third section, but preferably not the second section, comprises or consists of a material that allows a stretching of the relevant section in its circumferential or radial direction.

In some embodiments, the second section, and in particular its first subsection or only its first subsection, comprises or consists of a material which allows its expansion in its circumferential direction or radial direction.

In some embodiments, the first and/or the third section, but preferably not the second section, comprises a material that allows the respective section to be stretched in its circumferential direction or radial direction or it consists, individually or respectively, of such a material.

However, the elasticity of a section made of such a material is lower in a direction perpendicular to the circumferential direction (i.e. in the longitudinal direction of the prosthesis covering), for instance in the range between 0% up to 70% of the elasticity in circumferential direction. Elasticity may be achieved purely by way of example by fibres running in a wavy or zig-zag pattern in the circumferential direction together with e.g. fibres not running in a wavy or zig-zag pattern in the longitudinal direction.

In some embodiments, the first and/or third section comprises a different diameter and/or circumference than the second section, e.g., in the worn state, in the unworn state, lying on the table, or still in the package. In this, the diameter and/or circumference of the second section is preferably larger than the diameter and/or circumference of both the first and third section.

In several embodiments, the prosthesis covering may, in at least one (longitudinal) middle section, such as the second section, be wider than at least one section proximal of the middle section and wider than at least one section distal of the middle section.

In some embodiments, the shape of the prosthesis covering may initially be wider (or exactly one time wider) and then again narrower (or exactly one time again narrower) in the longitudinal direction.

The prosthesis covering may, in some embodiments, have its largest diameter and/or circumference in a middle section, e.g. the second section.

In several embodiments, the prosthesis covering may have its largest diameter and/or circumference in a middle section, e.g., the second section, in which it has no inner liner or silicone layer.

The diameters may be measured, for example, using rulers on the prosthesis covering lying flat on the table in a state in which it is not being stretched, expanded, etc. by external forces. The circumferences may, for example, be measured in such a state using a measuring tape, or may be estimated by doubling the diameter determined as above.

In several embodiments, the prosthesis covering, for example in a non-use state, comprises no curvature in its longitudinal direction, in particular none that is manufacture-related, for instance through the use of longer structures on a front side of the prosthesis covering as well as on a rear side (such as when wearing the prosthesis covering).

In some embodiments, the prosthesis covering comprises stiffening elements made of a strap/tape-like material that may be sewn, glued, or connected in another way to the outside or inside of the fabric or material of the prosthesis covering. Such a connection may be provided in sections or along the entire length of the stiffening member. Preferably, at least one middle section of the stiffening element (e.g., in the longitudinal direction thereof) is not connected to the prosthesis covering fabric, such as the second section.

Such stiffening members may be configured as a second subsection of the second section as described herein, and/or vice versa. Thus, what is mentioned for a second subsection herein may optionally also apply to the stiffening members, and vice versa.

In several embodiments, the wall of the second section—e.g. in a completely unfolded state thereof—does not comprise a variable radial thickness, especially of its wall, particularly not over the anterior side or the ventral side.

In some embodiments, the wall of the first and/or the third section—e.g. in a completely unfolded state thereof—has different radial thicknesses, especially of its wall, wherein preferably in particular the anterior or ventral side (when wearing the prosthesis covering) may be thicker than the posterior or dorsal side.

In some embodiments, the prosthesis covering does not comprise a bulge resulting from the use of different materials or material thicknesses, in none of the sections. In other embodiments, this is different, in particular with regard to the second section—this may have other or different wall thicknesses than the first and/or second section.

In several embodiments, the prosthesis covering or one or more of its sections mentioned herein are not made of a knitted, single-piece elastic tube. This applies particularly to the second section.

In some embodiments, the prosthesis covering does not comprise a fabric section which is stretchable in two, particularly perpendicular, directions. This applies particularly to the second section.

In several embodiments, the prosthesis covering does not comprise a material that restrains movement, optionally neither on the inner side nor on the outer side, in particular one that restrains a bending movement of the prosthesis covering wearer.

In some embodiments, the prosthesis covering comprises no plurality of annular protrusions, in particular not on or within the first, the second and/or third section.

In several embodiments, the prosthesis covering does not comprise, at least at its outer side, preferably at least not in an area which is assigned to the second section or is part thereof, any protrusions running longitudinally, which, being each (e.g. longitudinally) distributed around the circumference of the prosthesis covering, extend therealong.

In some embodiments, the outer skin of the prosthesis covering does not comprise any longitudinal protrusions—in particular due to the material thickness itself—which, being each (e.g. longitudinally) distributed around the circumference of the prosthesis covering, extending therealong. Notwithstanding this, however, an inner liner arranged radially inwardly relative to the outer skin, which may be present in the first and/or third section, may have a corrugated surface structure on its outer side. This may, e.g. due to the selected joining method for connecting the outer skin to the inner liner lying underneath it, also lay the outer skin in waves or folds.

In some embodiments, the outer skin is (an) at least or exactly three-layer.

In some embodiments, the prosthesis covering does not have, at least on its outer side, any longitudinal seams which extend in the longitudinal direction of the prosthesis covering. In other embodiments, the prosthesis covering comprises, at least on its outer side, a seam or joint or overlap that extends longitudinally relative to the prosthesis covering or to one or more of its sections, particularly the second section, preferably over first, second as well as third section, and particularly preferably over the entire length of the prosthesis covering or at least of the outer skin.

In some embodiments, the longitudinally extending seam, joint, or overlap, if any, is covered, on an inner side and/or on an outer side of the prosthesis covering, along its entire longitudinal extent or along a predominant section of its longitudinal extent (i.e., >50% of its length) by a tape or by a tape material.

The tape can be an adhesive tape.

The tape may be multi-layer, e.g., three-layer or more. The several layers may have different materials and may perform different functions. For example, a first layer may be an adhesive layer which may be liquefied, e.g. by hot air, in order to penetrate into the seam holes of the optional seam as well as into pores of the layers held together by the seam, e.g., that of the outer skin, which in turn may comprise a textile as the outermost layer, and to cure there. A second layer arranged over this adhesive layer may assume the function of a barrier layer and be for example impermeable to air. A third layer arranged over the second layer may have or represent a textile surface, wherein such a textile may be understood herein as a morphologically determinable, shaped structure of spinnable, length-limited fibers and (or) drawn, endless fibers having spinnability as a property.

The outer skin of the prosthesis covering, the outermost layer of the first section, of the second section and/or of the third section may also be exactly or at least three-layer. Thereby, a first layer and/or a third layer may each be a textile (identical or different from each other). A second layer disposed between them may thereby be a membrane.

The membrane may be or comprise, for example a polyurethane (PU), a polysulfone (PSU) or a polyethersulfone (PES).

The membrane may have a pore size of 20 μm to 70 μm. For its part, the membrane may consist of different layers, which may differ from each other in terms of the pore width and/or material (e.g. PU, PES, PSU) thereof, respectively.

In several embodiments, the outer skin comprises or consists of a membrane, but is not three-layer.

In some embodiments, the prosthesis covering does not comprise, at least at its outer side, any stabilizing element, in particular not a stabilizing element made of plastic, in particular not a rigid stabilizing element. This may exclude ribbon-like, pliable and/or flexible structures, such as e.g. the second subsection.

In several embodiments, the prosthesis covering does not comprise, or is not, a knitwear and/or knitted fabric.

In some embodiments, the prosthesis covering does not comprise a ring or a plurality of rings on its inner surface.

In several embodiments, there are no sticking or adhesive fibres integrated in the fabric, which protrude on the inner side of the prosthesis covering in order to ensure additional hold.

In some embodiments, the prosthesis covering is suitable and designed for creating a vacuum. Therefore, the material of the prosthesis covering, in particular of the first, second and/or third section, may be an airtight material, such that a created vacuum may be maintained at least for a specific period, for example, several hours or days. In several embodiments, the prosthesis covering does not comprise a valve, in particular not one which would lie in the wall of the first, second and/or third section.

In several embodiments, at least one diameter and/or circumference (or the average diameter or circumference, respectively) of the first section and/or the third section is, in an unstressed state or in a state of non-use of the prosthesis covering, smaller than a diameter and/or circumference (or the average diameter or circumference, respectively) of the second section. The diameter or the circumference of the second section may optionally be larger by between 5% and 20% than the diameter or circumference of the first and/or the third section. The above statements may apply in particular if the material of the sections under consideration (possibly after their destructive detachment from the prosthesis covering for the purpose of measuring) is/are smoothed out or drawn or pulled.

In some embodiments, at least one extension (or the average extension) of the second subsection in a stress-free state or a non-use state of the prosthesis covering is smaller than an extension (or the average extension) of the first subsection. The extension may be determined in the longitudinal direction of the prosthesis covering and thus correspond to the length of the subsection under consideration. The extension of the first subsection may optionally be between 5% and 20% greater than the extension of the second subsection. The extension of the first subsection may optionally be between 3 cm and 15 cm larger than the extension of the second subsection, preferably between 5 cm and 10 cm, most preferably between 6 cm and 9 cm. The above statements may apply in particular if the material of the subsections under consideration (possibly after their destructive detachment from the prosthesis covering for the purpose of measuring) is/are smoothed out or drawn or pulled.

In some embodiments, at least one extension (or the average extension) of the second section, of the first subsection and/or of the second subsection in a stress-free state or a non-use state of the prosthesis covering, is smaller than an extension (or the average extension) of the first and/or third section. The extension may be determined in the longitudinal direction of the prosthesis covering and thus correspond to the length of the subsection under consideration. The extension of the second section, the first subsection and/or the second subsection may optionally be between 50% and 500% greater than the extension of the first and/or the second section. The above statements may apply in particular if the material of the sections and subsections under consideration (possibly after their destructive detachment from the prosthesis covering for the purpose of measuring) is/are smoothed out or drawn or pulled.

In some embodiments, the wall thickness of the first section or of the third section is stronger, thicker, or greater in one area than in adjacent areas, in particular of the second section, e.g., in a stress-free state of the prosthesis covering or during normal use of the prosthesis covering.

In some embodiments, the second section, the first subsection and/or the second subsection—e.g. in a lower or a middle area, e.g. its lower half or lower third, (in the longitudinal direction of the prosthesis covering) of the third section—ends in a fastening section or extends to where the second section, the first subsection and/or the second subsection (preferably along the entire circumference of the first subsection or the second section) is connected (glued, sewn, welded, etc.) to the third section. n this way, the third section extends in the form of a protrusion—preferably on the inside and/or only on the inside—beyond the lower end of the second section, the first subsection and/or the second subsection towards the first section.

In several embodiments, this protrusion of the third section is, in whole or in sections, not connected (glued, sewn, etc.) to the second section, to the first subsection nor to the second subsection.

In some embodiments, the second section, in a fastening section, is connected to the third section, wherein the third section projects over the fastening section beyond the fastening section at least partially over the second section by protrusion of the third section. In this projecting section or protrusion, the material of the third section is not connected to the second section, such as by gluing, sewing, etc.

In some embodiments, the second section is connected, in a fastening section, e.g. a closed or annular fastening section, to the third section, wherein the third section projects by a protrusion at least partially beyond the fastening section of the third section towards the first section. In this projecting section or protrusion or sections thereof, the material of the third section is not connected to the second section, such as by gluing, sewing, etc.

In several embodiments, the third section extends along the inside of the second section by a protrusion, through which it protrudes beyond the fastening section between the third and second section.

After acquisition of the prosthesis covering, the protrusion disclosed herein may be tailored by the wearer of the prosthesis covering or by the prosthetist to the individual needs of the patient. Geometric features of the prosthesis, which may be associated with hard materials and/or edges, can thus be covered by the softer and/or thicker material of the third section when the prosthesis covering is worn, while the covering may be cut away where it can have no such benefit for reasons of weight saving and greater wearing comfort.

In some embodiments, the lamellae are provided in the form of waves or corrugations.

In several embodiments, the first section is narrower (longitudinally) and/or longer (circumferentially or unwound) than the third section.

In some embodiments, the second section has a larger circumference or is unwound larger than the first section and/or than the second section.

In several embodiments, the second section has a circumference that increases in a longitudinal direction of the second section or increases in a longitudinal direction of the second section when unwound.

In some embodiments, the second section and/or its subsections or materials thereof have a lower extensibility than the first section and/or than the third section or materials thereof, e.g., in a longitudinal direction of the prosthesis covering.

In strength theory, to which reference is made herein, stretchability is understood to be the property of a material to change its shape when a force is applied. Stretchability indicates how far a material can be extended without breaking or tearing.

The following applies for the stretching:

$$ε = \frac{Δ L}{{L}_{0}}$$

Elasticity, as used herein, means the property of a body or material to change shape when a force is applied thereon and to return to its original shape when the applied force is removed (example: spring).

The modulus of elasticity, as used herein, is defined, for example, as the slope of the graph in the stress-stretching diagram at uniaxial load with infinitesimal change in distortion at zero stress. Most materials have a(n) (at least small) linear range, this is also called Hooke's range. The following applies:

$$E = \frac{σ}{ε} = const$$

Here, σ (=force/surface) denotes the mechanical stress (normal stress, not shear stress) and ε=ΔL/L₀ denotes the stretching. The stretching is the ratio of the change in length ΔL=L−L₀ to the original length L₀. The unit of the modulus of elasticity is that of a stress:

$$E in \frac{N}{{mm}^{2}} , in Si - Einheiten : E in \frac{N}{{m}^{2}} ( Pascal )$$

The modulus of elasticity is called material constant, since by using it and the transverse Poisson's numbers the law of elasticity is established. However, the modulus of elasticity is not constant with respect to all physical quantities. It depends on various environmental conditions such as, for example, temperature or humidity. Therefore, comparable conditions are assumed herein when determining the modulus of elasticity.

In several embodiments, the prosthesis covering comprises at least or exactly one second section on each of its medial side and its lateral side.

In several embodiments, the prosthesis covering comprises a layer, e.g. an outer skin, extending over all three sections. In some embodiments, this layer is not glued or otherwise connected to a second layer in the area of the second section, in particular not over its entire circumference.

In some embodiments, the first section comprises an inner liner on the inside of the outer skin or consists of the outer skin and the inner liner as well as optionally of ends of the second subsection or ribbons.

In several embodiments, the inner liner is cylindrical (when the prosthesis covering is held accordingly) or a differently shaped hollow body with a through-passage direction in the longitudinal direction of the prosthesis covering.

The inner liner may be circumferential, and it may optionally have a length which is greater than its wall thickness or its average wall thickness.

The inner liner may, in several embodiments, be made, for example, of silicone or comprise silicone.

In several embodiments, the wall thickness of the inner liner, in an unloaded state of the prosthesis covering, is e.g. in a range of 3 mm to 5 mm, preferably 4 mm.

The outer skin, or its material, may be airtight in some embodiments. It may, optionally, be or comprise a membrane fabric. It may optionally be two-, three-, or multi-layer.

In particular, the outer skin and the inner liner may be made of, or comprise, different materials.

The outer skin and inner liner may be glued or otherwise joined to each other in the area of the first section and/or of the third section. The gluing or other connection may extend over the entire length of the first or third section or the inner liner present there, or over only a part thereof (in the longitudinal direction of the prosthesis covering).

In some embodiments, an end section of the subsection, which may have or be, for example, a lateral ribbon as described herein and/or a medial ribbon, may be incorporated into this connection or gluing, for example by common connection or gluing. The first and/or the third section may thus become, in sections, an exactly three-layer adhesive section or at least three-layer adhesive section, but may have one layer less at other locations.

In several embodiments, the outer skin does not comprise a circumferential seam (i.e., no seam that extends in the circumferential direction), which may be associated with manufacturing and durability advantages.

In some embodiments, the outer skin is continuous—at least in the longitudinal direction. It may be made e.g. of a textile, membrane, film, etc., and/or of combinations thereof.

Unlike the first section and/or the third section, the second section in some embodiments preferably does not comprise an inner liner or has an inner liner that differs from that of the first and/or third section in material and/or thickness.

The inner liner of the first section and of the third section correspond to each other in material and/or thickness in some embodiments.

In some embodiments, the inner liner of the first section and/or the third section comprises—in the circumferential direction—a longer outer side than inner side, which is not solely due to the fact that there is a larger diameter on the outer side of the inner liner than on the inner side thereof. Rather, the longer outer side is explained by the fact that this is shaped, for example, in waves, folds, etc., whereas the inner side is made smooth or less wavy, folded, etc.

The wavy, curvy, folded, raised, etc. outer side may be glued to the outer skin such that said outer skin is also in waves, folds, etc. in those areas in which it is connected, e.g. glued, to the inner liner.

In some embodiments, the maximum circumference of the second section is 10%, 20%, 30%, 50%, 100%, 150% or more of the circumference in the area of the first section and/or in the area of the third section.

In some embodiments, only a proximal or distal section of the inner liner is directly or indirectly connected or glued to the outer skin, while the distal or proximal section is not.

In some embodiments, the circumference at the proximal end of the inner liner of the first section is wider or longer than the circumference at the opposite, distal end of this inner liner. This inner liner may thus have a slightly conical shape and/or an inclination of its outer surface towards a middle line (e.g., in a cross-section). The inclination may be up to 5° or more, preferably between 0.5° and 3°. The latter may have manufacturing advantages. The inclination may optionally apply only to the proximal section of this inner liner.

The length of the inner liner of the first section may be, for example, between 90 cm and 120 cm, the circumference may be, for example, between 250 cm and 450 cm, and/or the diameter may be, for example, between 80 cm and 145 cm.

In several embodiments, the circumference at the proximal end of the inner liner of the third section is wider or longer than the circumference at the opposite, distal end of this inner liner. This inner liner may thus have a slightly conical shape and/or an inclination of its outer surface towards a middle line (in a cross-section). The inclination may be up to 5° or more, preferably between 0.5° and 3°. The latter may have manufacturing advantages.

The length of the inner liner of the third section may be, for example, between 140 cm and 250 cm, the circumference may be e.g. between 210 cm and 370 cm, and/or the diameter may be, for example, between 67 cm and 118 cm.

In several embodiments, the circumference at the proximal end of the outer skin (e.g. prior to its optional connection to the inner liners, etc.) is wider or longer than the circumference at the opposite, distal end of the outer skin.

In some embodiments, the wall thickness or average wall thickness of the second section is less, at least in sections or continuously, than the wall thickness or average wall thickness of the first and/or the third section.

In several embodiments, the material thickness or average material thickness of the outer skin is less, at least in sections or continuously, than the material thickness or average material thickness of the inner liner of the first and/or third section.

In some embodiments, the material thickness or average material thickness of the outer skin is at least 1.5 mm less, at least in sections or continuously, than the material thickness or average material thickness of the inner liner of the first and/or third section.

In several embodiments, the outer skin is the first subsection. What is stated here for the outer skin may apply undiminished to the first subsection, and/or vice versa.

The present invention also encompasses the fact that it is not true or not only true that the material or a material of the second section has a greater modulus of elasticity than one or more of the materials of the first section and/or of the third section, but that alternatively or in addition, the second section has a greater modulus of elasticity in exactly one or at least one direction than the first section and/or the third section. According to the present invention, all features and embodiments mentioned herein may also be read for or combined with this embodiment.

Some or all embodiments according to the present invention may comprise one, several or all of the advantages mentioned above and/or in the following.

It may be of advantage that the prosthesis covering, when it is put on the patient in order to bridge a joint, may provide a high level of comfort when being worn, due to a material reserve which the prosthesis covering provides for the wearer, for example, on its anterior or ventral side by the second section. Therefore, depending on the design of the second section, no force or only a lower amount of force is required in comparison to common prosthesis coverings, when bending the joint covered or bridged by the prosthesis covering, e.g. the artificial knee when walking.

A rubbing on the skin or on a layer lying under the prosthesis covering, e.g. a liner, which is caused by stretching the prosthesis covering on the extensor side of the joint followed by a contraction of the prosthesis covering over or on the prosthesis or the skin of the patient, may in some embodiments also be advantageously prevented or reduced as the second section lying on the extensor side of the joint is not elastic or only elastic to a comparatively low degree and therefore exerts no pressure onto the skin and/or does not lie on or contact the skin.

Additionally, due to the particular selection of the herein-described Young's moduli of the prosthesis covering, it may be ensured that the prosthesis covering does not exert pressure onto the underlying structures or tissues, independent of a bending of the joint, at least in the area of the section which is non-elastic, less elastic or only elastic to a comparatively low degree.

The second subsection may—if provided—advantageously prevent the second section from extending or stretching to the maximum, already when the prosthesis covering is pulled on or put on, and thus unintentionally minimizing or eliminating the material reserve which is also responsible for wearing comfort.

### BRIEF DESCRIPTION OF THE DRAWINGS

The present invention is exemplarily explained with regard to the accompanying drawing in which same reference numerals refer to the same or similar components. In the figures, the following applies:

FIG. 1 shows a prosthesis covering according to the present invention in a first embodiment;

FIG. 2 shows a prosthesis covering according to the present invention in a second embodiment;

FIG. 2a shows the lower section of the prosthesis covering according to the present invention of FIG. 2 in a partial sectional view;

FIG. 3 shows a prosthesis covering according to the present invention in a third embodiment;

FIG. 3a shows the prosthesis covering according to the present invention of the third embodiment;

FIG. 3b shows a prosthesis covering according to the present invention in a fourth embodiment;

FIG. 3c shows a prosthesis covering according to the present invention in a fifth embodiment;

FIG. 3d shows a prosthesis covering according to the present invention in a sixth embodiment;

FIG. 3e shows a prosthesis covering according to the present invention in a seventh embodiment;

FIG. 4 shows a prosthesis covering according to the present invention in an eighth embodiment;

FIG. 5 shows a prosthesis covering according to the present invention in a ninth embodiment; and

FIG. 5a shows a section through the prosthesis covering according to the present invention of FIG. 5 along a line B-B of FIG. 5.

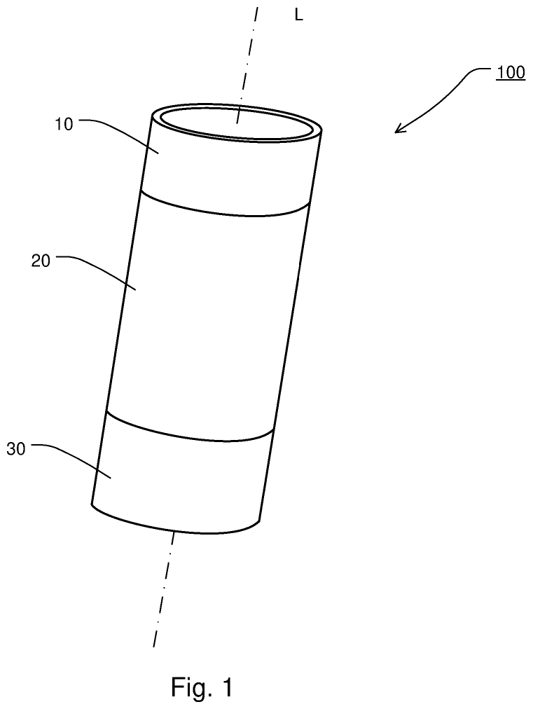

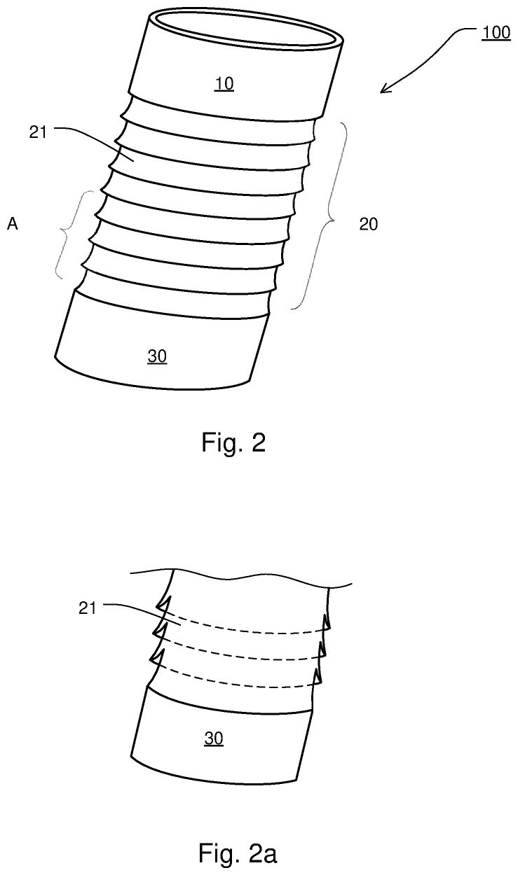

FIG. 1 shows a prosthesis covering 100 according to the present invention in a first embodiment in a side view with a slight perspective from above.

The prosthesis covering 100 comprises a first section 10, a second section 20 and a third section 30, or alternatively consists thereof. Its longitudinal direction or longitudinal axis is indicated with L. The prosthesis covering 100 extends in an axial direction. The transverse direction or the radial direction extends perpendicular to the longitudinal direction L.

Thereby, the second section 20 is arranged between the first section 10 and the third section 30. In the example of the figures the first section 10 and the third section 30 are not in contact with each other. As in FIG. 1, they are optionally in at least a radial layer, not in contact with each other.

The second section 20 comprises a higher E-Modul (Young's modulus) than the first section 10 and/or than the third section 30.

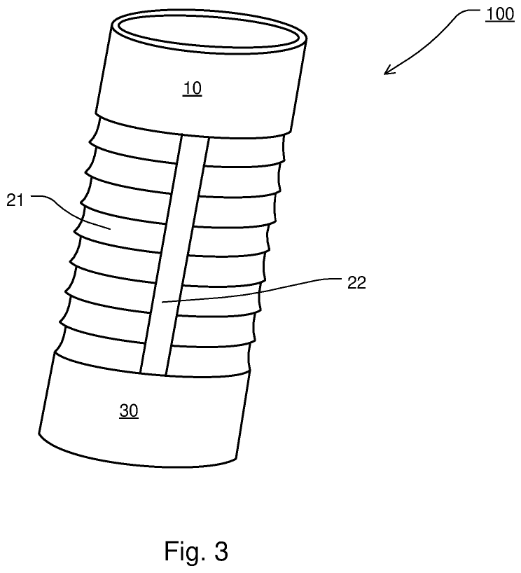

FIG. 2 shows a prosthesis covering 100 according to the present invention in a second embodiment.

Thereby, in a first subsection 21 thereof, the second section 20 is folded lamella-like along its entire circumference. In FIG. 2 there are shown, for example, eight lamellas, any other number is equally encompassed by the present invention.

The lamellas serve as an example of a material reservoir, in particular in the longitudinal direction of the second section 20 (see the line L in FIG. 1). When the prosthesis covering 100 is put over a joint, for example the artificial knee joint, and the patella or the artificial patella of the wearer of the prosthesis covering 100 is pointing to the left with regard to FIG. 2 (with the popliteal fossa (hollow of the knee) on the right side of the prosthesis covering 100 with respect to FIG. 2), the second section 20 with a higher Young's modulus than sections 10 and/or 30 may be extended/lengthened by unfolding, e.g. over the knee.

Hereby, the patient feels no pressure on the skin which would result from the tension of an elastic material in the second section 20. The second section 20 (in comparison to a lower Young's modulus of the first section 10 and/or the third section 30) does not allow such pressure to occur. Instead of building up pressure, it simply lengthens (extends) in the longitudinal direction due to the lamella-like structure provided therefore.

FIG. 2a shows the lower section of the prosthesis covering 100 according to the present invention of FIG. 2 in a partial sectional view A. The section shown in FIG. 2a is indicated with the reference numeral A in FIG. 2.

It is visible, that the material of the first subsection 21 of the second section 20, which is folded in lamellas, lies on top of each other due to a double over. In the state of the prosthesis covering 100 shown in FIG. 2 and FIG. 2a, the material is still doubled over on itself or still comprises doubled over parts, however, the main extension of them lies (more or less) parallel to the longitudinal axis of the prosthesis covering 100, in the embodiment shown, however, not perpendicular to it.

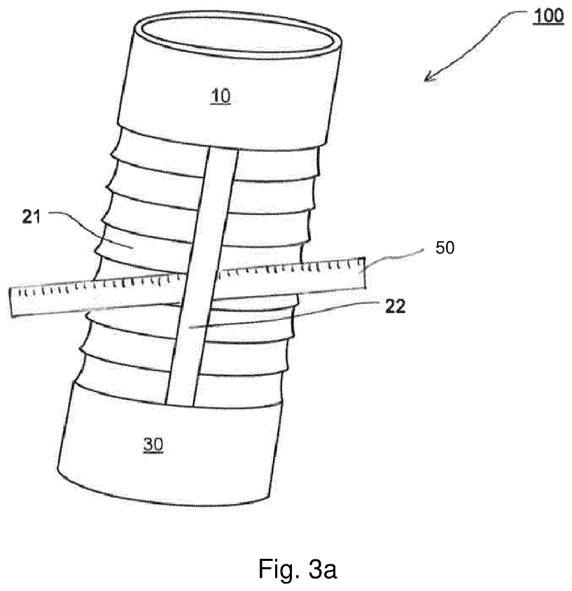

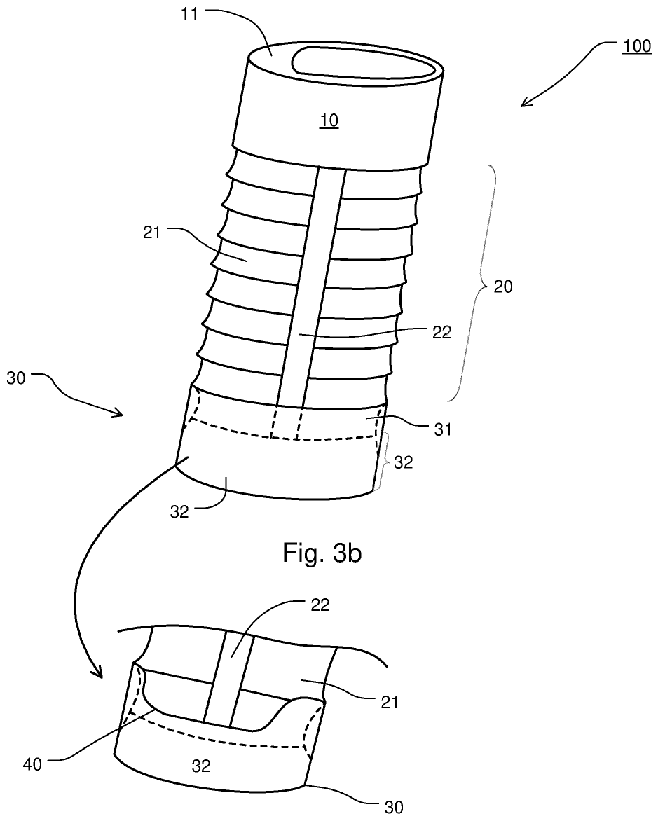

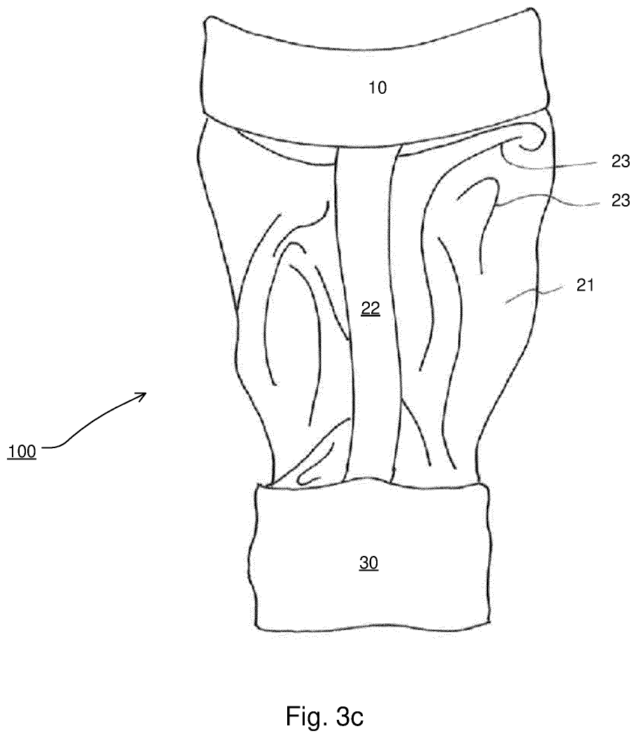

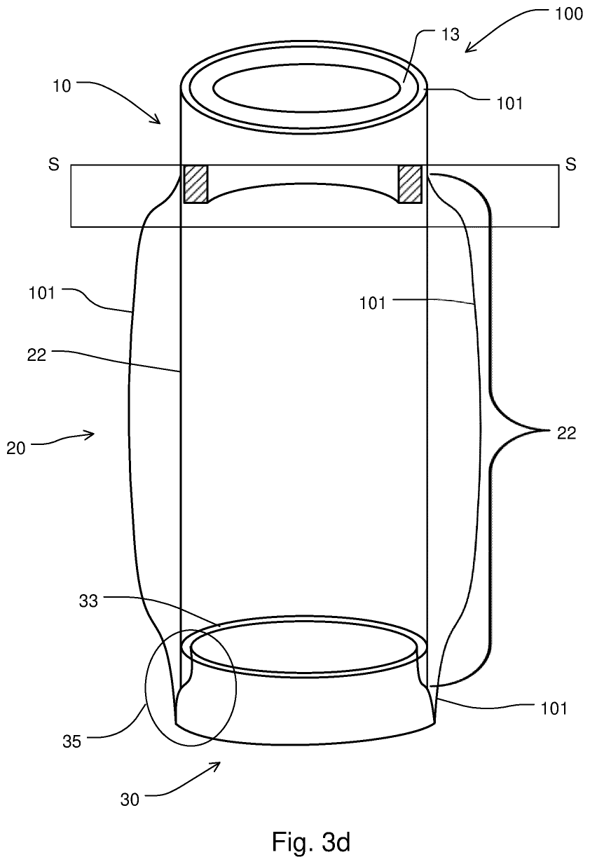

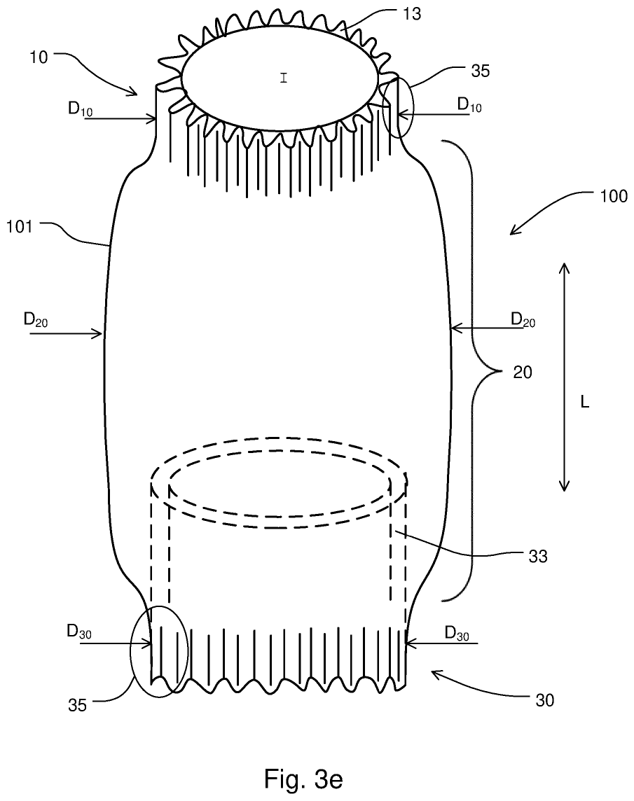

FIG. 3 shows a prosthesis covering 100 according to the present invention in a third embodiment.

The prosthesis covering 100 differs from the one in FIG. 2 preferably only in having a second subsection 22.

The second subsection 22 connects the first section 10—directly or indirectly—with the third section 30. It may ensure that the distance between the first section 10 and the third section 30 (in longitudinal direction L) does not exceed a maximum distance or a desired maximum distance. It may further ensure that the material reservoir of the second section 20 does not unfold while the prosthesis covering is being pulled on or put on and is no longer available to the patient during later flexion movements.

Unlike the first subsection 21, the length or the visible or unfolded length of which may be increased by unfolding, the second subsection 22 does not comprise a material reserve. Its length is unchangeable, however, within its elasticity or its non-elasticity.

Thereby, the second subsection 22 may be less elastic in comparison to the elasticity of the first section 10 and/or the third section 30. It may optionally be non-elastic. The same applies to the second section 20 in longitudinal direction, which is, due to the second subsection 22, less elastic than the first and/or the third section 10, 30.

In FIG. 3 a second subsection 22 can be seen. However, two or more of such second subsections 22 may be provided distributed over the circumference of the prosthesis covering 100, for instance a second subsection 22 on the medial side of the prosthesis covering 100 and a further subsection 22 on the lateral side of the prosthesis covering 100. Further second subsections 22 may be provided, it may also be the case that such further subsections 22 are not provided.

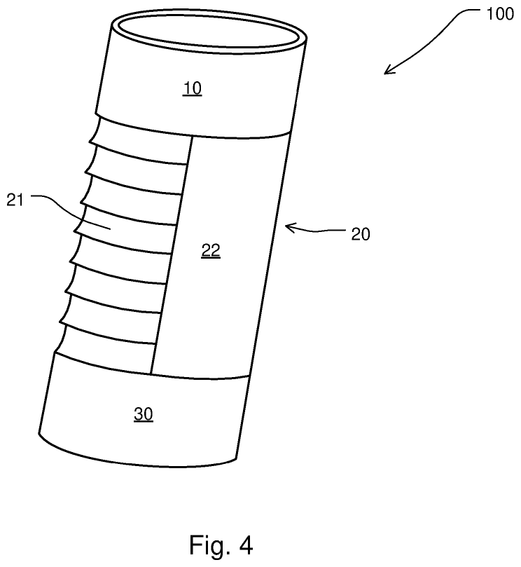

Also, the width of the second subsection 22 may vary. In FIG. 3 the second subsection 22 is shown comparatively narrow and/or as a ribbon. Alternatively, it may be wider than shown in FIG. 3. It may constitute e.g. up to 100°, 120°, 140°, 160° or 180° or more of the circumference of the prosthesis covering 100. The latter embodiment is shown in FIG. 4.

The second subsection 22 preferably rests loosely on the first subsection 21 in at least one section (on the outside or the inside of the first subsection 21). In this section in which it rests loosely, it is not for example glued or sewn or otherwise connected to the underlying first subsection 21.

The loosely resting section can be 30%, 40%, 50%, 60%, 70%, 80% or more of the length or the visible length of the second subsection 22.

Due to the fact that the aforementioned section of the second subsection 22 rests loosely on the first subsection 21, e.g. a finger or a flat hand may be pushed through between the second subsection 22 and the first subsection 21. This embodiment allows the first subsection 21, which lies under the second subsection 22, to unfold freely at least in the area of the loosely resting section, should this be necessary when wearing the prosthetic covering 100. The relative movement between the second subsection 22 and the first subsection 21, made possible by the loosely resting section, contributes advantageously to said free unfolding.

Although it is mentioned above that the second subsection 22 rests on the first subsection 21, this is not intended to restrict the invention (as shown in FIG. 3 or disclosed in any other embodiment or combination of features). In fact, the second subsection 22 may alternatively or additionally also lie under the first subsection 21, or between two layers of the first subsection 21. Hence, the loosely resting section of the second subsection 22 could lie under the first subsection 21 or between two layers of the first subsection 21 and not be connected to neither the upper nor the lower layer. Thus, relative movement between the second subsection 22 and both layers of the first subsection 21 is possible.

FIG. 3a shows the prosthesis covering 100 of the third embodiment according to the present invention.

In FIG. 3a, compared to the illustration of the prosthesis covering 100 in FIG. 3, the loosely resting section is emphasized by showing a drawing ruler 50 which is put between the first subsection 21 and the second subsection 22, wherein the subsection 21 is shown lying below the second subsection 22, which is reverse in any other embodiments. There, the first subsection 21, no matter how it is designed, lies above the second subsection 22 and thus optionally also above the loosely resting section.

The drawing ruler 50 is pushed or slid through under the second subsection 22 and above the first subsection 21. The loosely resting section, i.e. that section in which the first subsection 21 is not connected to the second subsection 22 by a material connection (gluing, textile welding or the like) or by a force-fit or form-fit connection (stitching, hook-and-loop fastener or the like) or connected in any other way, permits such a push-through.

It has been shown through non-public experiments by the inventor that a loose section can significantly improve the comfort of the patient or user of the prosthesis covering.

FIG. 3b shows a prosthesis covering 100 according to the invention in a fourth embodiment.

It can be seen that the wall thickness of the first section 10 can optionally be thicker in an area 11 than in adjacent areas, wherein the area 11, for example when the prosthesis covering 100 is worn, may face forward (ventrally).

FIG. 3b further shows, and to be considered independently of the reinforcement of the wall in the first section 10, an optional manner of a fastening of the second section 20, the first subsection 21 and/or the second subsection 22 to the third section 30.

As can be seen from FIG. 3b, the second section 20, the first subsection 21 and/or the second subsection 22—e.g. in a lower or a middle area (in the longitudinal direction of the prosthesis covering 100) of the third section 30—end in a connecting section or fastening section 32, in which the second section 20, the first subsection 21 and/or the second subsection 22 (preferably along the entire circumference of the first subsection 21 or the second section 20) are connected (glued, sewn, welded, etc.) to the third section 30. In this manner, the third section 30 protrudes beyond the lower end of the second section 20, of the first subsection 21 and/or of the second subsection 22 towards the first section 10 over the fastening section 32. The section thus protruding is referred to herein as the protrusion 31.

If this protrusion 31 of the third section 30 is not connected (glued, sewn, etc.) in whole or in sections to the second section 20, to the first subsection 21 and/or to the second subsection 22, it can be cut or trimmed to size by the user of the prosthesis covering 100 in a simple manner in order to suit their needs. For example, the protrusion 31 may be trimmed along a cut line 40 which is to be determined by the user e.g. in order to fit the upper edge of a prosthesis.

This is shown in FIG. 3b in a sectional representation of the prosthesis covering 100.

It does not matter whether the fastening section 32, in which the second section 20 or its subsections 21, 22 are connected to the third section 30, extends to a lower end of the third section 30 or is merely a strip or otherwise shaped area beyond which the third section 30 is in turn not connected (glued, welded, etc.), e.g. downwardly with respect to FIG. 3b, to the second section 20 in a direction away from the first section 10. Each of these embodiments is encompassed by the present invention.

It is further encompassed by the present invention that the protrusion 31 of the third section 30 abuts the second section 20 externally or internally, which may apply to any embodiment.

FIG. 3c shows a prosthesis covering 100 according to the present invention in a fifth embodiment, in which the lamellae of previous embodiments are in the form of waves or corrugations. By way of example, two areas in which the second section 20 is present in a doubled manner are denoted by the reference numeral 23.

In FIG. 3c, it is further shown that, the first section 10 may in any embodiment be narrower (longitudinally) and/or longer (circumferentially or when unwound) than the third section 30.

In FIG. 3c, it is further shown that the second section 20 may have a larger circumference or be larger when unwound than the first section 10 and/or the third section 30.

FIG. 3c further shows that the second section 20 may have a circumference that increases in a longitudinal direction of the second section 20 or that increases in a longitudinal direction of the second section 20 when unwound.

The second subsection 22, which is also shown on the outside in FIG. 3c, lies in other embodiments on the inside of the second section 20.

FIG. 3d shows a prosthesis covering 100 according to the present invention in a sixth embodiment. It is shown in section in an area S, schematically simplified and partially cut below this area.

In the sixth embodiment, the prosthesis covering 100 comprises a layer extending over all three sections 10, 20 and 30, which covers the prosthesis covering 100 here on its outer side, which is why it is referred to herein—not restrictively—as outer skin 101. In other embodiments than the one shown here, this layer extending in a longitudinal direction of the prosthesis covering 100 over all sections 10, 20 and 30 completely or at least in sections, respectively, may be present on an inner side or in a central area of the wall of the prosthesis covering 100 (relative to its thickness or material thickness) instead of being present on the outside.

Although the outer skin 100 ends, as an example of the aforementioned layer, at the proximal as well as at the distal end of the prosthesis covering 100 in the exemplary embodiment shown in FIG. 3d, it is also encompassed by the present invention that the outer skin 101 extends over or into all three sections 10, 20 and 30, but ends—e.g. a few millimeters or centimeters—before the proximal and/or before the distal end of the prosthesis covering 100 (in its longitudinal direction).

The first section 10 comprises an inner liner 13 on the inside of the aforementioned outer skin 101 or consists of the outer skin 101 and the inner liner 13. The position of the outer skin 101 and the inner liner 13 relative to each other, according to which the outer skin 101 rests, as its name suggests, on the outside of the inner liner 13 is as mentioned above, however, only exemplarily. Alternatively, the material or element referred to herein as inner liner 13 could rest on the outside of the outer skin 101 and/or radially with respect thereto. The latter would then be an inner skin, and the inner liner 13 rather a sheathing, so to speak.

The inner liner 13 may be cylindrical (by appropriately holding the prosthesis covering), or it may be a differently shaped hollow body with a through-direction in the longitudinal direction of the prosthesis covering.

The inner liner 13 may be circumferential or closed, and it may optionally have a length that is greater than its wall thickness or its average wall thickness.

For example, the inner liner 13 may be or comprise silicone, or may be or comprise another material. The inner liner 13 may be designed to advantageously conform for example to the patient's skin in a smooth and preferably airtight manner.

The wall thickness of the inner liner 13 in a non-stressed state of the prosthesis covering may be for example in a range from 3 mm to 5 mm, preferably 4 mm.

The outer skin 101 or its material may be airtight. It may be, optionally, a membrane fabric. It may optionally be two-, three- or multi-layer.

In particular, the outer skin 101 and the inner liner 13 may be of different materials.

Outer skin 101 and inner liner 13 may be glued or otherwise connected to each other in the area of the first section 10. The gluing or other connection may extend along the entire length of the first section 10 or over only a section thereof (in the longitudinal direction of the prosthesis covering).

Incorporated into this connection or gluing may be an end section of the subsection 22, which may have or be, for example a lateral ribbon as described herein (right in FIG. 3d) and a medial such ribbon (left in FIG. 3d), e.g., by connecting or gluing them together. More or less than two such ribbons may also be provided. Their arrangement is not limited to medially or laterally or to 180° offset from each other with respect to the circumference of the prosthesis covering 100. For example, the end of the subsection 22 may be interposed between the outer skin 101 and the inner liner 13 as shown in FIG. 3d, and all three elements 101, 13 and 22 may be connected, in this case glued, together in a three-layer adhesive section as adhesive section 35 (or other joining section) being shown for the connection comprising the elements 101, 33 and 22 Alternatively, the subsection 22 is connected or glued to the outer skin 101, and the outer skin 101 is connected to the inner liner 13, but the subsection 22 is not directly connected to the inner liner 13.

Exemplarily, the outer skin 101 does not comprise a circumferential seam over its entire length, but at least in the transitions between the first and second sections 10, 20 and/or between the second and third sections 20, 30, which may be associated with manufacturing and durability advantages.

The outer skin 101 may thus—at least in the longitudinal direction—be of continuous design made of an uninterrupted textile, such a membrane, foil, etc.

In some arbitrary embodiments, such as that of FIG. 3d, the second section 20, which is arranged between the first section 10 and the third section 30 and thus represents their connection, comprises the subsection 22, which consists of or at least comprises the two ribbons shown here with a view to, respectively, their narrow side and the outer skin 101 associated with them radially on the outside, i.e. to their outer sides.

It can be seen that in this embodiment the outer skin 101 has at least two diameters and/or circumferences, namely a first diameter and/or circumference in the area of the first section 10 and a second diameter and/or circumference in the area of the second section 20, wherein the first diameter and/or circumference is smaller than the second.

When reference is made herein to the first diameter or circumference being smaller than the second diameter or circumference, this may refer to a relaxed state of the prosthesis covering 100, such as when the prosthesis covering 100 is not being worn but is, for example, still in the closet or packaging.

The first diameter or circumference may e.g. therefore be smaller than the second diameter or circumference because for manufacturing the prosthesis covering 100 less material was used for the outer skin 101 in the area of the first diameter/circumference than in the area of the second diameter, and the circumference is thus smaller with the same wall thickness.

However, the first diameter or circumference may e.g. also therefore be smaller than the second diameter or circumference because for manufacturing the prosthesis covering 100, the outer skin 101 was permanently pre-stretched in the area of the second diameter or circumference, unlike in the area of the first diameter or circumference. The pre-stretching may involve plastic deformation, which is why the outer skin 101 may have a smaller wall thickness in the area of the second diameter or circumference than in the area of the first diameter or circumference.

The first diameter or circumference may also therefore be smaller than the second diameter, because when manufacturing the prosthesis covering 100, the outer skin 101 was doubled or placed in waves in the area of the first diameter or circumference and connected in this state, for example glued, to the inner liner 13 and/or 33. In this way, the outer skin 101, were it to be released from its connection or gluing and subsequently smoothed, would have a first circumference in the area of the first diameter which is or may be as large as the second circumference. In the connected state, i.e. when the prosthesis covering 100 is manufactured, as shown for example in FIG. 3d, the outer skin 101 is namely unable to unfold freely due to the doubling, gathering, corrugation, etc. in the first section 10 and to the fact that it is connected in the area of the first section 10 to the inner liner 13 and/or 33, which is of smaller circumference and/or diameter than the outer skin 101 in the area of its second diameter. Its diameter and/or circumference thus corresponds approximately to the outer diameter or outer circumference of the inner liner 13; such a case is shown in FIG. 3e.

The choice of different diameters or circumferences results in or leads to a shape of the prosthesis covering 100 as shown in FIG. 3d and FIG. 3e. A narrower first section 10 is followed by the further or wider second section 20 and by the third section 30 which is optionally again narrower than the second section 20, possibly even of the same diameter or circumference as the first section 10.

As in other embodiments disclosed herein, the subsection 22 may ensure that the distance between the first section 10 and the third section 30 (in the longitudinal direction L) does not exceed a maximum distance or a desired maximum distance between the first section 10 and the third section 30.

In this, the second section 20 is preferably, due to its subsection 22, less elastic than the first section 10 and/or than the third section 30. It may optionally be non-elastic, particularly with respect to its second subsection 22.

What is set forth herein for the inner liner 13 or the first section 10 may apply undiminished also for an inner liner 33, which is optionally shown on an inner side of the outer skin 101 in the area of the third section 30 in FIG. 3d, and/or for the third section 30.

Unlike the first section 10 and/or the third section 30, the second section 20 preferably does not comprise an inner liner, in particular not in the sense of the inner liner 13 or 33.

FIG. 3e shows a prosthesis covering 100 according to the present invention in a seventh embodiment in side view with a slight perspective from above.

As can be seen from FIG. 3e, the prosthesis covering 100, like that of FIG. 3d, has in its second section 20 a circumference and/or a diameter D20 which is larger than the circumference and/or the diameter D10 of the first section, and which is also larger than the circumference and/or the diameter D30 of the third section 30.

Purely optionally, the prosthesis covering 100 of FIG. 3e comprises one or several of the features in any combination described herein.

In particular, the prosthesis covering 100 of FIG. 3e may optionally comprise any features of the embodiment of FIG. 3d.

Further, the prosthesis covering 100 of FIG. 3e or any other embodiment may optionally comprise the following features, each alone or in any combination with each other and/or with other features as described herein.

Thus, the inner liner 13 in FIG. 3e, and here exemplarily also the inner liner 33, are manufactured wavy on their outside, with a preferably smooth inside, as the view into the interior I of the prosthesis covering 100 shows.

The wavy outer surface may be glued to the outer skin 101 such that it is also wavy in those areas in which it is for example glued to the inner liner 13 or 33. This is different in the free area, i.e. where the outer skin 101 is not glued as described above. There it may lie free of waves, hence, this is the reason why the outer skin 101 has a smaller diameter (see FIG. 3e) in the area where it is glued to one of the inner liners 13, 33, although there—in the unfolded state—it optionally has the same circumference.

The maximum circumference in the area of the first section 10 and/or in the area of the third section 30 is, for example, 20 cm, while the maximum circumference in the area of the second section 20 is, for example, 50 cm.

As can be seen from the upper or proximal section of the inner liner 33 shown by the dashed line, the inner liner 13 or 33 may be only partially connected or glued to the outer skin 101. A section of the inner liner 13, 33 may be free from gluing.

The optional subsection 22 of FIG. 3d is not shown nor outlined herein.

In some embodiments, the outer skin 101 is the first subsection 21. What is set forth herein for the outer skin 101 may apply undiminished to the first subsection 21, and in several embodiments, the reverse is also true.

FIG. 4 shows a prosthesis covering 100 according to the present invention in an eighth embodiment.

The prosthesis covering 100 corresponds to the one shown in FIG. 3, except that there is only one second subsection 22, which extends over 180° of the circumference of the prosthesis covering 100.

The first subsection 21 may be optionally supplemented to 360° with the second subsection 22, as shown by way of example in FIG. 4.

The first subsection 21 may cover optionally 360° of the circumference of the extremity or of the prosthesis covering 100. The second subsection 22 may lie on or adjacent to the first subsection 21, optionally at its inner or outer surface.

As shown in FIG. 4 by way of example, the first subsection 21 may constitute or cover 180° of the circumference of the prosthesis covering 100. It may alternatively also be wider than shown in FIG. 4. Thus, it may constitute e.g. up to 240° or more of the circumference of the prosthesis covering 100.

In any embodiment according to the present invention, the first subsection 21 may cover between 40% and 70% of the circumference, more preferably a part of the circumference, constituting between 55% and 65% of the circumference, most preferably 60% of the circumference.

The percentages mentioned above may optionally refer to the unstressed state of the prosthesis covering 100, for example, before use, e.g. in the shipping packaging, or it may refer to a state of use, for example, on the extremity.

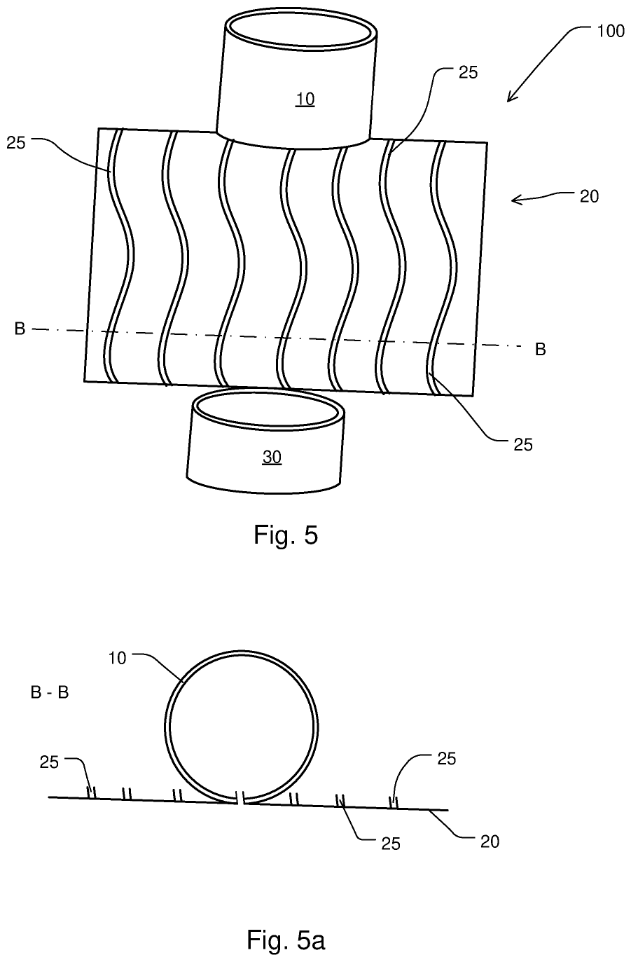

FIG. 5 shows a prosthesis covering 100 according to the present invention in a ninth embodiment in a partial sectional view.

Only the second section 20 is cut and unfolded or unwound, but not the first section 10 nor the third section 30. The view to the drawing plane of FIG. 5 shows the inner side of the second section 20, which would be in contact with the skin of the patient during use of the prosthesis covering 100 or, in any case, would be facing it.

An elevated or prominent structure 25 may be recognized, which extends away from the inner side of the second section 20, and which, during use of the prosthesis covering 100, extends radially into its interior.

The structure 25 may preferably extend in a longitudinal direction of the prosthesis covering 100. It serves to prevent the second section 20 from lying completely planar on the patient's body. Benefited by the distance, the structure 25 keeps open between the inner side of the second section 20 and the patient's body (e.g. the skin), a negative pressure between the inner side of the second section 20 and the patient's body may be established by suitable negative pressure devices without any relevant niche formations.

FIG. 5a shows the single cut through the prosthesis covering 100 according to the present invention of FIG. 5 along a line B-B of FIG. 5.

### LIST OF REFERENCE NUMERALS

100 prosthesis covering
101 outer skin
10 first section
11 area
13 inner liner
20 second section
21 first subsection
22 second subsection
23 doublings, doubled area
25 elevated structure
30 third section
31 protrusion
32 fastening section
33 inner liner
35 sticking area
40 cutting line
50 drawing ruler
D10 diameter of the first section
D20 diameter of the second section
D30 diameter of the third section
B-B cutting line
I interior of the prosthesis covering
L longitudinal direction

1. A prosthesis covering having a closed cross-section
wherein the prosthesis covering comprises along its longitudinal extension at least a first section, a second section and a third section,
wherein the second section comprises a larger circumference or diameter than both the first section and also the third section and/or wherein the second section is made of, or comprises, a material comprising a higher E-Modul (Young's modulus) than one or several of the materials of the first section and/or the third section, and
wherein the second section is arranged between the first section and the third section.

2. The prosthesis covering according to claim 1, having a layer or outer skin which extends continuously over the first section, the second section and the third section, wherein the layer or outer skin is connected, in the first section and/or in the third section, but not in the second section, to an inner liner, preferably directly or indirectly and/or in the radial direction.

3. The prosthesis covering according to claim 1, wherein the first section, the second section and/or the third section is made of, or comprises, an airtight material.

4. The prosthesis covering according to claim 1, wherein the first inner liner and/or the second inner liner is made of, or comprises, silicone.

5. The prosthesis covering according to claim 1, wherein the first subsection of the second section or the outer skin is not elastic in a circumferential direction.

6. The prosthesis covering according to claim 1, wherein the first section and/or the third section are elastic.

7. The prosthesis covering according to claim 1, wherein the second section comprises at its inner side at least one ribbon-shaped structure as second subsection.

8. The prosthesis covering according to claim 1, wherein the second section is connected to the third section in a fastening section, and wherein the third section partially protrudes the second section beyond the fastening section towards s the first section by a protrusion of the third section, in which material of the third section is not connected to the second section.

9. A prosthesis, in particular leg prosthesis or knee prosthesis, which is at least partially covered or overlaid by a prosthesis covering according to claim 1.

10. A set having a prosthesis and a prosthesis covering according to claim 1.

11. A prosthesis covering, in particular according to claim 1, having a closed cross-section,
wherein the prosthesis covering comprises along its longitudinal extension at least a first section, a second section and a third section,
wherein the second section is made of, or comprises, a material comprising a higher E-Modul (Young's modulus) than one or several of the materials of the first section and/or the third section, and
wherein the second section is arranged between the first section and the third section.
